(19) United States
(12) Patent Application Publication
(10) Pub. No.: US 2015/0066010 A1
(43) Pub. Date: Mar. 5, 2015

(54) EXPANDABLE MESH PLATFORM FOR CARDIAC ABLATION

(71) Applicant: Cook Medical Technologies LLC, Bloomington, IN (US)

(72) Inventors: Tyler E. McLawhorn, Winston-Salem, NC (US); Vihar C. Surti, Winston-Salem, NC (US)

(73) Assignee: COOK MEDICAL TECHNOLOGIES LLC, Bloomington, IN (US)

(21) Appl. No.: 14/284,944

(22) Filed: May 22, 2014

Related U.S. Application Data
(60) Provisional application No. 61/827,380, filed on May 24, 2013.

(51) Int. Cl. A61B 18/14; A61B 18/12

(52) U.S. Cl. CPC A61B 18/1492; A61B 18/1206; A61B 2018/00375
USPC 606/34

(57) ABSTRACT

An ablation device and methods for using the same. The ablation device has an inner shaft, an outer shaft, a mesh, a conductive coating on the mesh, and a compression mechanism. The inner shaft is disposed within the outer shaft and the compression mechanism moves the inner shaft relative to the outer shaft. The mesh expands when the compression mechanism moves the inner shaft proximally relative to the outer shaft. Electrical energy is delivered to the conductive coating to ablate tissue proximate the conductive coating.

### RELATED APPLICATIONS

The present patent document claims the benefit of the filing date under 35 U.S.C. §119(e) of Provisional U.S. Patent Application Ser. No. 61/827,380 filed May 24, 2013, which is hereby incorporated by reference.

### FIELD

This invention relates generally to medical devices for ablating tissue in a body lumen. More particularly, this invention relates to a system for ablating tissue around an ostium of a blood vessel.

### BACKGROUND

Atrial fibrillation is a common form of cardiac arrhythmia that can lead to a multitude of health problems including chronic heart failure and stroke. During atrial fibrillation (AF) the electrical impulses originating in the pulmonary veins become disorganized and generate irregular impulses of the ventricles. One of the current treatments of AF is atrial ablation via an intracardiac catheter. This ablation disrupts the irregular electrical impulses stemming from the pulmonary veins. To ensure adequate ablation, the physician will ablate circumferentially around where the pulmonary veins enter the left atrium. To do this, the physician has to ablate multiple places with each ablation partially overlapping adjacent ablations to form a continuous ablation line. Ensuring that the ablation line is continuous can be difficult as there is no direct visual feedback and the ablation sites themselves do not show up under either fluoroscopy or ultrasound. If the ablation line is not continuous, the ablation procedure can potentially be ineffective. Additionally, as the atrium is continuously moving, ensuring adequate contact between the tissue and the electrode can be difficult.

It would be beneficial to have a system capable of ablating around the ostium in a single session.

### SUMMARY

Embodiments of the invention include a medical device for ablating tissue around an ostium. The medical device comprises a first longitudinal member having a first longitudinal member distal end, a second longitudinal member having a second longitudinal member distal end, a mesh, and a conductive coating. The second longitudinal member is axially movable from a proximal position to a distal position. The mesh has a distal end attached to the second longitudinal member and a mesh proximal end attached to the first longitudinal member. The mesh comprised of a plurality of flexible non-conductive filaments woven together, the mesh being expandable from an unexpanded configuration to an expanded configuration by axially moving the first longitudinal member and the second longitudinal member relative to each other.

In another embodiment, a medical device for ablating tissue around an ostial vessel comprises a sheath, a shaft, a mesh, and a conductive coating. The sheath has a longitudinal lumen with an inside diameter. The shaft is disposed within the longitudinal lumen of the sheath and is moveable relative to the lumen from a proximal position to a distal position. The mesh is attached to a distal end of the shaft and is mesh biased to a shape having a mesh outside diameter greater than the inside diameter of the sheath and comprises a plurality of flexible non-conductive filaments. The conductive coating is disposed on an outer surface of the mesh.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 illustrates a cross section of a heart with an ablation device disposed in the right atrium.

FIG. 2 illustrates a cross section of the heart with the ablation device disposed in the left atrium.

FIG. 3 illustrates a cross section of the heart with the ablation device in an expanded configuration.

FIG. 4 illustrates a cross section of an ablation device.

FIG. 5 illustrates the ablation device of FIG. 4 in an expanded configuration.

FIG. 5a illustrates the ablation device of FIG. 5 in a head on view.

FIG. 6 illustrates the ablation device of FIG. 5 further expanded.

FIG. 6a illustrates the ablation device of FIG. 6 in a head on view.

FIG. 7 illustrates an ablation device having a conical expanded mesh.

FIG. 8 illustrates another embodiment of an ablation device.

FIG. 9 illustrates an embodiment of a bipolar ablation device.

FIG. 9a illustrates the ablation device of FIG. 9 in a head on view.

FIG. 10 illustrates an embodiment of an ablation device in a vessel.

FIG. 11 illustrates an embodiment of an ablation device having a conical mesh.

FIG. 12 illustrates a proximal end of an ablation device having an energy source.

The drawings are not necessarily to scale.

### DETAILED DESCRIPTION

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Detailed Description does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

In the following discussion, the terms “proximal” and “distal” will be used to describe the opposing axial ends of the inventive ablation device, as well as the axial ends of various component features. The term “proximal” is used in its conventional sense to refer to the end of the ablation device (or component thereof) that is closest to the operator during use of the ablation device. The term “distal” is used in its conventional sense to refer to the end of the ablation device (or component thereof) that is initially inserted into the patient, or that is closest to the patient during use. For example, an ablation device may have a proximal end and a distal end, with the proximal end designating the end closest to the operator, such as a handle, and the distal end designating an opposite end of the ablation device. Similarly, the term “proximally” refers to a direction that is generally towards the operator along the path of the ablation device and the term “distally” refers to a direction that is generally away from the operator along the ablation device.

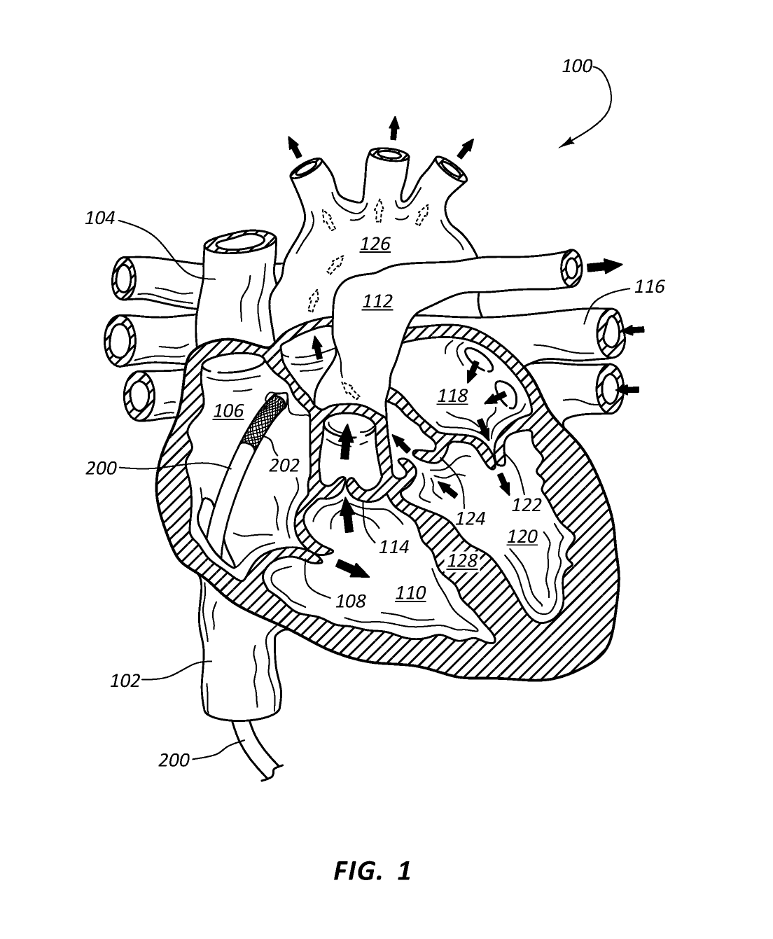

FIG. 1 is a simplified cut-away view of a heart 100 showing the various chambers and vessels of the heart 100. Blood (depicted by arrows) flows to the heart 100 from the body through the inferior vena cava 102 and the superior vena cava 104 into the right atrium 106. From the right atrium 106 blood flows through the tricuspid valve 108 into the right ventricle 110. From the right ventricle 110 blood is pumped into the pulmonary artery 112 through the pulmonary valve 114. The blood flows into the lungs from the pulmonary artery 112 and returns via the pulmonary vein 116. From the pulmonary vein 116, blood collects in the left atrium 118 and flows into the left ventricle 120 through the mitral valve 122. The right ventricle 110 and the left ventricle 120 are separated by a septum 128. Blood flows from the left ventricle 120 through the aortic valve 124 to the aorta 126 where it is delivered to the body.

A distal end of an ablation device 200 having an expandable mesh 202 is disposed in the right atrium 106. In this example the ablation device 200 has been delivered through the lower vena cava 102 and the distal end of the ablation device 200 extends from the lower vena cava 102 into the right atrium 106. The proximal end of the ablation device 200 extends from the lower vena cava 102 to a location outside the patient's body. For example a patient may have a small incision made in a vein of the lower extremities which is then used to access the vascular system and guide a guidewire (not shown) to the right atrium 106. With the guidewire in place, the ablation device 200 may be advanced over the guidewire to the right atrium 106. In some embodiments the ablation device 200 may be delivered through the superior vena cava 104.

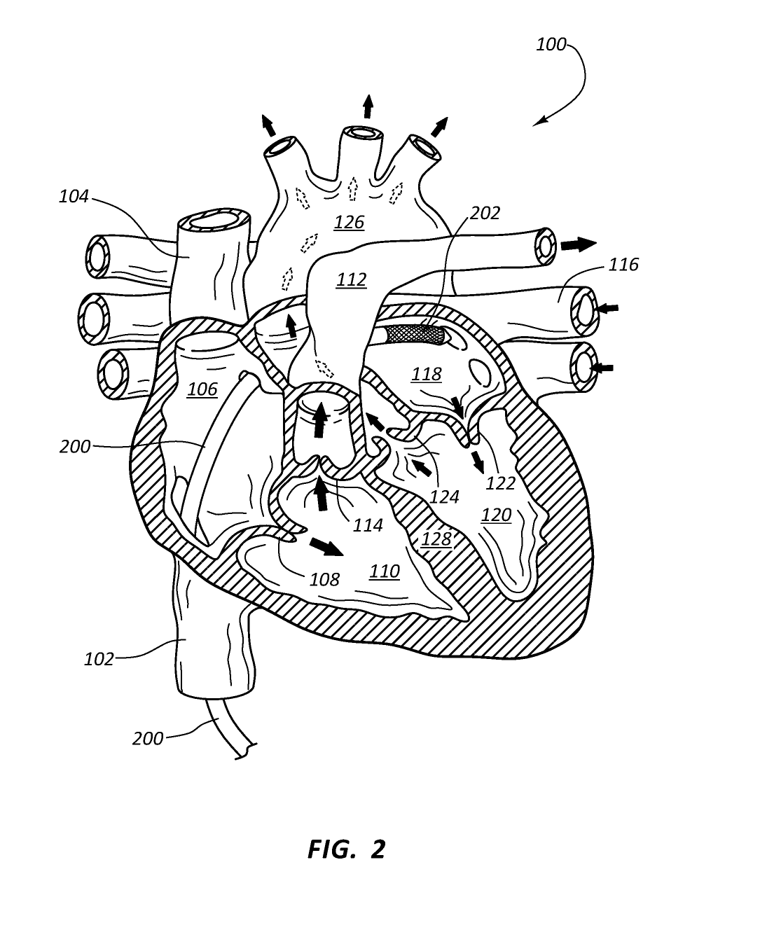

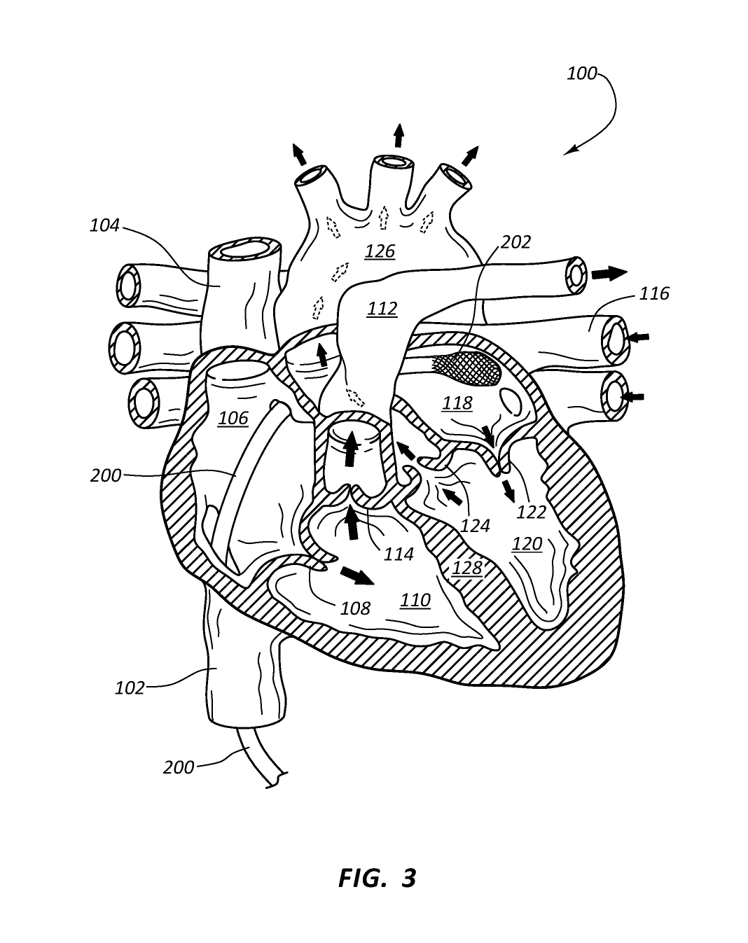

FIG. 2 illustrates the heart 100 of FIG. 1 with the distal end of the ablation device 200 being advanced into the left atrium 118. Because there is septum separating the right atrium 106 from the left atrium 118, the septum must be punctured to allow the distal end of the ablation device 200 to pass into the left atrium 118. The septum may be punctured using commonly available techniques known to those of ordinary skill in the art. Once the distal end of the ablation device 200 is within the left atrium 118 the expandable mesh 202 is expanded as shown in FIG. 3. The expandable mesh 202 may be expanded into a pancake structure, a disk structure, an umbrella structure, or other structures as will be described hereafter. Because the expandable mesh 202 is flexible, it conforms to the wall of the left atrium 202. Additionally, the mesh is porous, allowing blood to continue to flow through the mesh during use. Flexible electrodes (not illustrated) placed on a distal end of the expandable mesh also conform to the wall of the left atrium 202 such that energy may be efficiently delivered from the electrode to the wall of the left atrium 202 to ablate tissue.

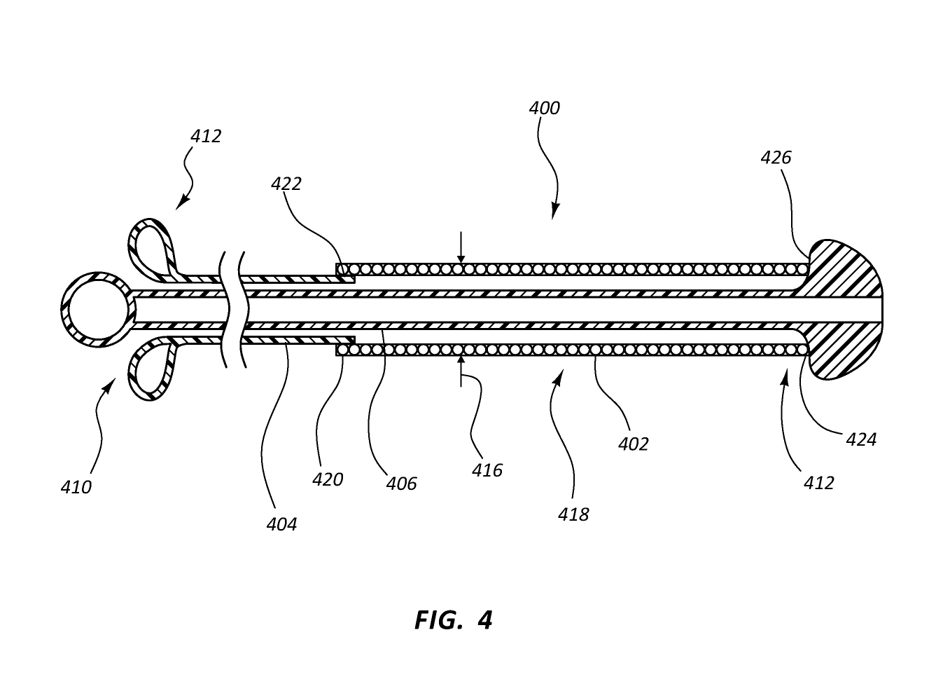

FIG. 4 illustrates a distal end of an embodiment of an ablation device 400 having an expandable mesh 402 in accordance with the present invention. The expandable mesh 402 may be comprised of a plurality of nonconductive filaments woven together in a cylindrical shape. The expandable mesh 402 is operably connected to an inner shaft 406 and an outer shaft 408. The inner shaft 406 may have at least one inner lumen sized to receive a guidewire. The at least one inner lumen may extend the entire length of the inner shaft. In use, the guidewire may be advanced to the ablation site and the ablation device 400 the advanced over the guidewire. The expandable mesh 402 may be secured at a proximal end 420 to a distal end 422 of the outer shaft 408 and at a distal end 424 to a distal end 426 of the inner shaft 406. The expandable mesh 402 may be secured to the shafts 406, 408 using common techniques such as bands, adhesives, thermal bonding, or other techniques as known in the art.

In some embodiments, the inner shaft 406 is coaxially positioned within the outer shaft 408 as shown in FIG. 4. The expandable mesh 402 expands and collapses by longitudinal movement of the inner shaft 406 relative to the outer shaft 408 as explained in more detail below. A control handle 410 is provided at a proximal portion 412 of the ablation device 400. The control handle 410 is operable to control the movement of the inner shaft 406 and the outer shaft 408 relative to one another. The control handle 410 may be any type of handle that is operable to control the movement of the inner shaft 406 relative to the outer shaft 408 and need not have the structure illustrated in FIG. 4.

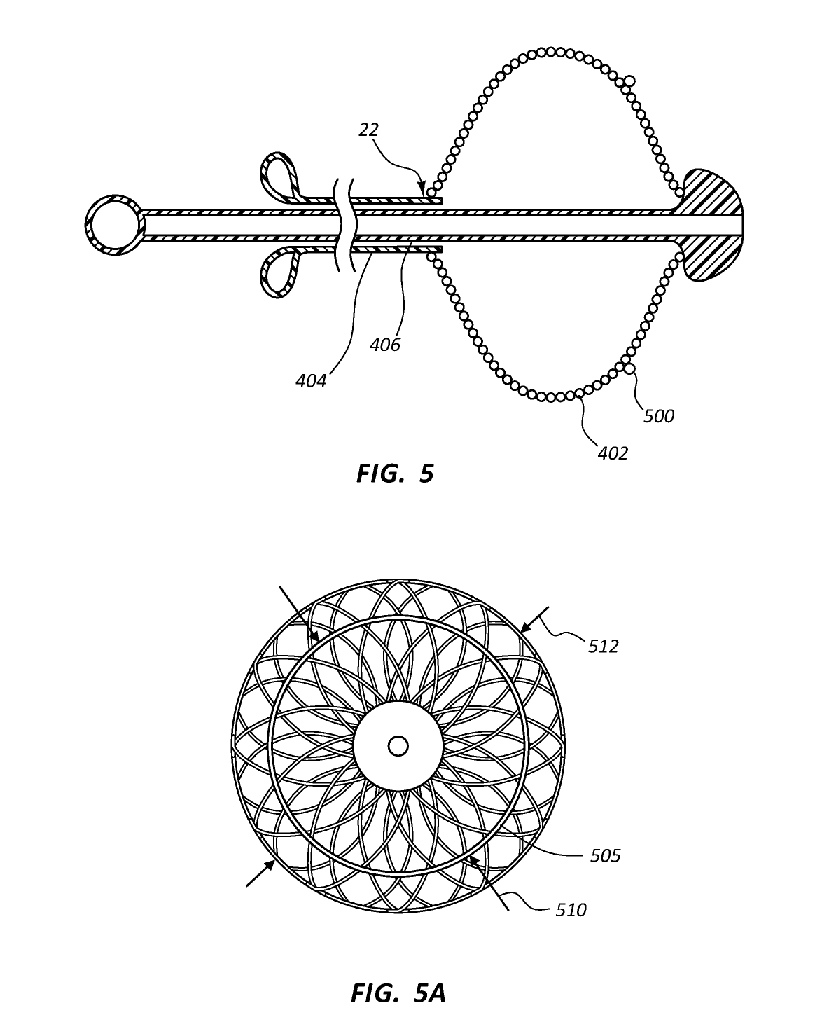

The nonconductive filaments may be formed from a nonconductive material such as a polyolefin, a fluoropolymer, a polyester, for example, polypropylene, polytetrafluoroethylene, polyvinylidene fluoride, polyethylene terephthalate (PET), and combinations thereof. Other materials known to one skilled in the art may also be used to form the filaments, provided that they enable the expandable mesh to be changeable from the collapsed configuration of FIG. 4 and the expanded configuration of FIG. 5 in response to the inner shaft 406 moving relative to the outer shaft 408.

Relative movement between the inner shaft 406 and the outer shaft 408 causes the expandable mesh 402 to change between a collapsed configuration shown in FIG. 4, and an expanded configuration shown in FIG. 5. The shape of the expanded configuration will vary depending on the amount of movement of the inner shaft 406 and the outer shaft 408 and the configuration of the expandable mesh 402. The expandable mesh 402 in the unexpanded configuration has a first outside diameter 416 and the expandable mesh 402 in the expanded configuration extends beyond the first outside diameter 416 at a middle segment 418. The unexpanded configuration may be used to deliver the distal end of the ablation device 400 to a treatment site within a patient and for repositioning the ablation device 400 within a patient to provide treatment to additional sites if needed.

FIG. 5 illustrates a distal end of an embodiment of an ablation device 400 having the expandable mesh 402 in an expanded configuration. In this embodiment the outer shaft 404 has been advanced distally relative to the inner shaft 406 decreasing the distance between the distal end 422 of the outer shaft 404 and the distal end 424 of the inner shaft 406. The resulting longitudinal compression of the expandable mesh 402 causes the expandable mesh 402 to expand radially into the shape shown in FIG. 5. Also shown is a flexible conductive coating 500 applied to a distal side of the expandable mesh 402 in a circumferential ring 508. In some embodiments the flexible conductive coating 500 may not extend along the entire circumference of the expandable mesh 402 and may be a circumferential segment of conductive coating 500. In other embodiments the flexible conductive coating 500 may extend from near an end of the mesh to an area near a mid-point of the expandable mesh to cover a circumferential portion of the expandable mesh. The flexible conductive coating 500 may comprise a conductive ink. The flexible conductive coating 500 is operably coupled to a conductor that extends to an energy source. The energy source provides a source of energy for ablating tissue proximate the flexible conductive coating 500.

The flexible conductive coating 500 may be arranged in other patterns for ablating different shapes. For example, the flexible conductive coating 500 may be arranged in a zig-zag pattern or have longitudinally extending components to ensure contact with the wall. The flexible conductive coating 500 may be applied only to individual filaments, leaving the mesh porous where the flexible conductive coating 500 is applied. In other embodiments, the flexible conductive coating 500 may cover a gap between adjacent filaments increasing the surface area of the flexible conductive coating 500. A base layer may be provided between the conductive coating 500 and the expandable mesh 402. The base layer may be used to span the space between adjacent filaments or to increase the adhesion of the flexible conductive coating 500 to the expandable mesh 402. By way of non-limiting example, the base layer may comprise silicone, silanes, chlorinated polyolefins, thiolated polymers, organosilanes, organotitanates, zirconates, and zircoaluminates.

FIG. 5a illustrates a front view of the ablation device 400 of FIG. 5 showing the flexible conductive coating 500 wrapping around the circumference of the expandable mesh 402 in a circumferential ring 508. The circumferential ring 508 of flexible conductive coating 500 has a diameter 510 that is dependent on a diameter 512 of the expandable mesh 402. As the diameter 512 of the expandable mesh 402 increases, the diameter 510 of the circumferential ring of flexible conductive coating 500 increases as well.

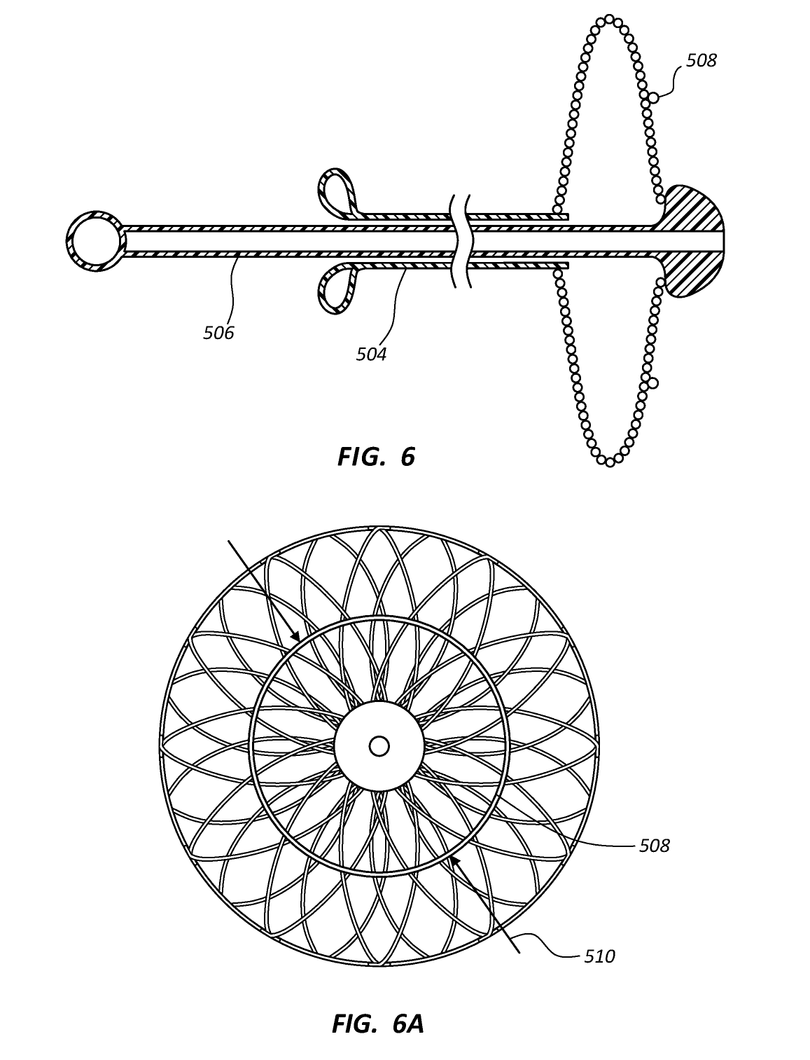

The expandable mesh of FIG. 5 may be further expanded by advancing the outer shaft 404 relative to the inner shaft 406. FIG. 6 illustrates the expandable mesh 402 being expanded further. Outer shaft 404 has been advanced relative to the inner shaft 406 causing the expandable mesh 402 to form a pancake shape. As shown in FIG. 6a the diameter 510 of the circumferential ring 508 of flexible conductive coating 500 is greater than it was in FIG. 5. This allows the expandable mesh 402 to have an adjustable size for varying geometries. If a larger ostium is being ablated the expandable mesh 402 may be expanded larger. For a smaller ostium it may be beneficial to keep the diameter 510 of the circumferential ring 508 of conductive coating 500 smaller.

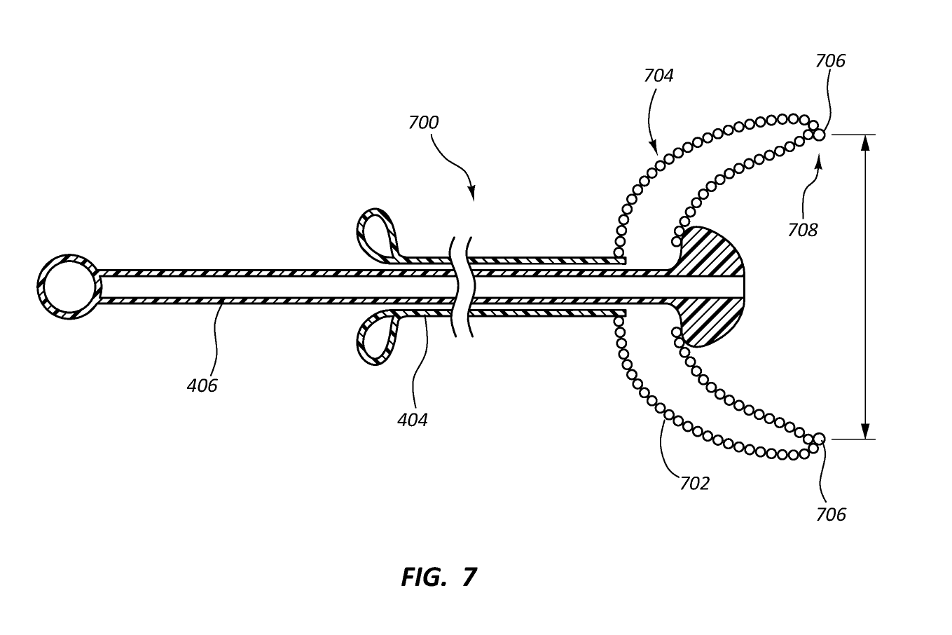

FIG. 7 illustrates another embodiment of an ablation device 700. This embodiment is substantially similar to the previously described embodiment of FIG. 4, with the exception of the shape of the expandable mesh 702 and the flexible conductive coating 706. In this embodiment the expandable mesh 702 is formed into an inverted cone like shape 704. The expandable mesh 702 may be formed into an inverted cone like shape 704 by bringing together a distal end 710 of the expandable mesh 702 and a proximal end 712 of the expandable mesh 702 to form a pancake shape, and then folding the resulting pancake shape distally. The flexible conductive coating 706 is placed on a base 708 of the inverted cone like shape 704. The diameter 710 of the base 708 of the inverted cone like shape 704 may be adjusted by movement of the inner shaft 406 relative to the outer shaft 404 as described previously.

It may be preferable to approach the ostium of a blood vessel from within the vessel itself instead of from the heart as described previously. In such embodiments the ablation device will pass out of the vessel and into the heart, where the expandable mesh is expanded. The ablation device is then retracted until the proximal side of the ablation device contacts the wall near the ostium.

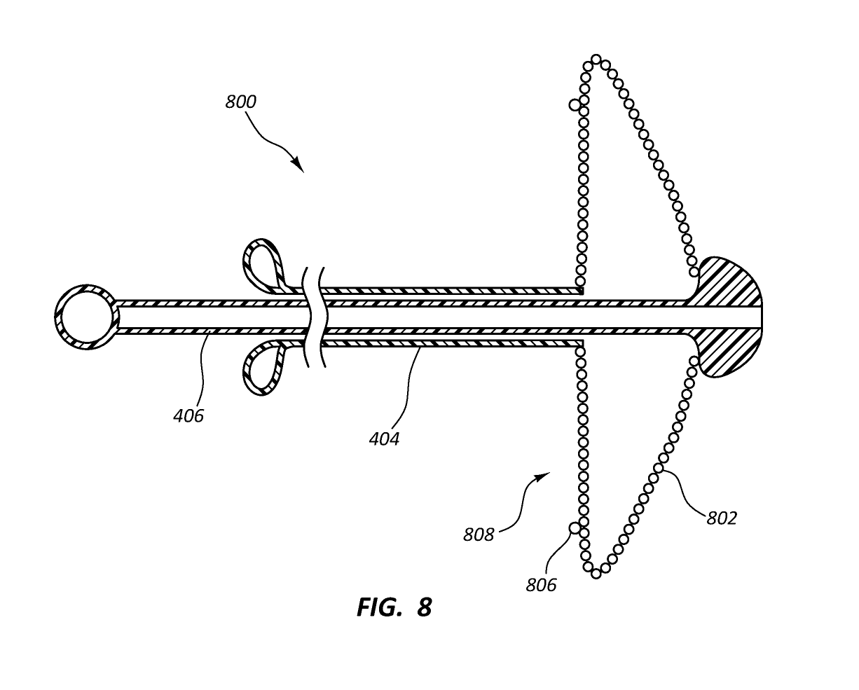

FIG. 8 illustrates an embodiment of an ablation device 800 suitable for abating an ostium of a blood vessel from within the blood vessel. This embodiment is substantially similar to the previously described embodiment of FIG. 4, with the exception of the placement of the flexible conductive coating 806. The flexible conductive coating 806 is placed on a proximal side 808 of the expandable mesh 802 in a circumferential strip that forms a ring shape when the expandable mesh is expanded, as shown in FIG. 8. The distal end of the ablation device 800 may be advanced into the patients left atrium 118 and the expandable mesh 802 is expanded. The distal end of the ablation device 800 is then retracted until the expandable mesh 802 and the flexible conductive coating 806 contact the wall of the atrium 118. Energy is then delivered to the flexible conductive coating 806 through a conductor and the wall is ablated. The expandable mesh 802 is then returned to its unexpanded state and may be removed from the atrium 118.

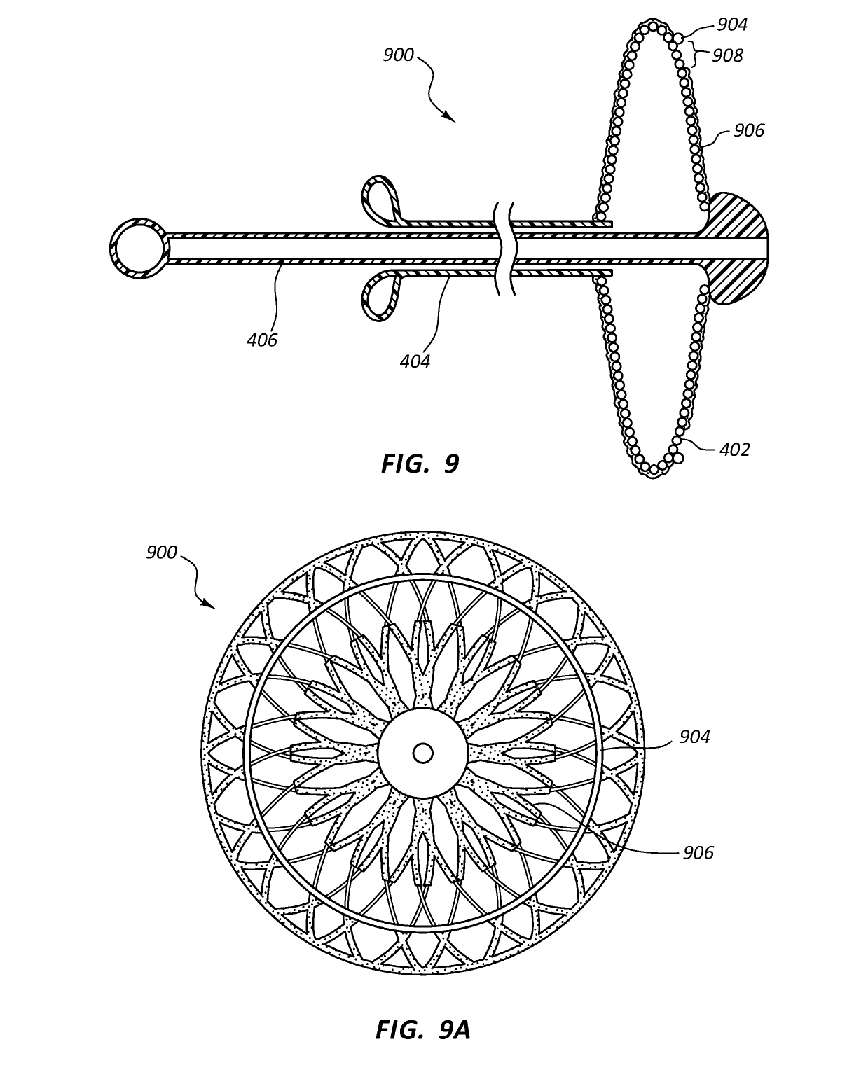

FIG. 9 and FIG. 9a illustrate another embodiment of an ablation device 900. This embodiment is substantially similar to the previously described embodiment of FIG. 4, with the exception of the configuration of the flexible conductive coating. In this embodiment, the ablation device 900 has a first conductive coating 904 and a second conductive coating 906 to form a bipolar configuration. The first conductive coating 904 is in electrical communication with a first pole of a power source and the second conductive coating 906 is in electrical communication with a second pole of a power source. The first conductive coating 904 and the second conductive coating 906 are separated by a nonconductive annular region 908 of the expandable mesh 402. The ablation device functions in the same manner as the embodiment of FIG. 4, changing size and shape as the inner shaft 406 is moved relative to the outer shaft 404. Because the flexible conductive coating covers a large portion of expandable mesh, the flexible conductive coatings 904, 906 typically coat only the filaments leaving space between individual filaments. The spaces allow blood to continue to flow through the expandable mesh while in use.

A thermistor 910 may be disposed near the nonconductive annular region 908. The electrical resistance of the thermistor 910 may vary depending on the temperature of tissue near the thermistor. The electrical resistance of the thermistor 910 may be measured to provide a measurement of the temperature of the tissue near the expandable mesh 406 during ablation. Other types of temperature sensors may be used to measure the temperature such as resistance temperature detector (RTD) or thermocouple.

To ablate tissue in the embodiment of FIG. 9, the expandable mesh is expanded and pressed against the wall of the heart. Energy is delivered to the first conductive coating 904 and the second conductive coating 906 from the power source. The first conductive coating 904 acts as a first pole and the second conductive coating 906 acts as the second pole of the power source. With the expandable mesh pressed against the wall, ablation will occur in tissue proximate the nonconductive annular region between the poles of the energy source.

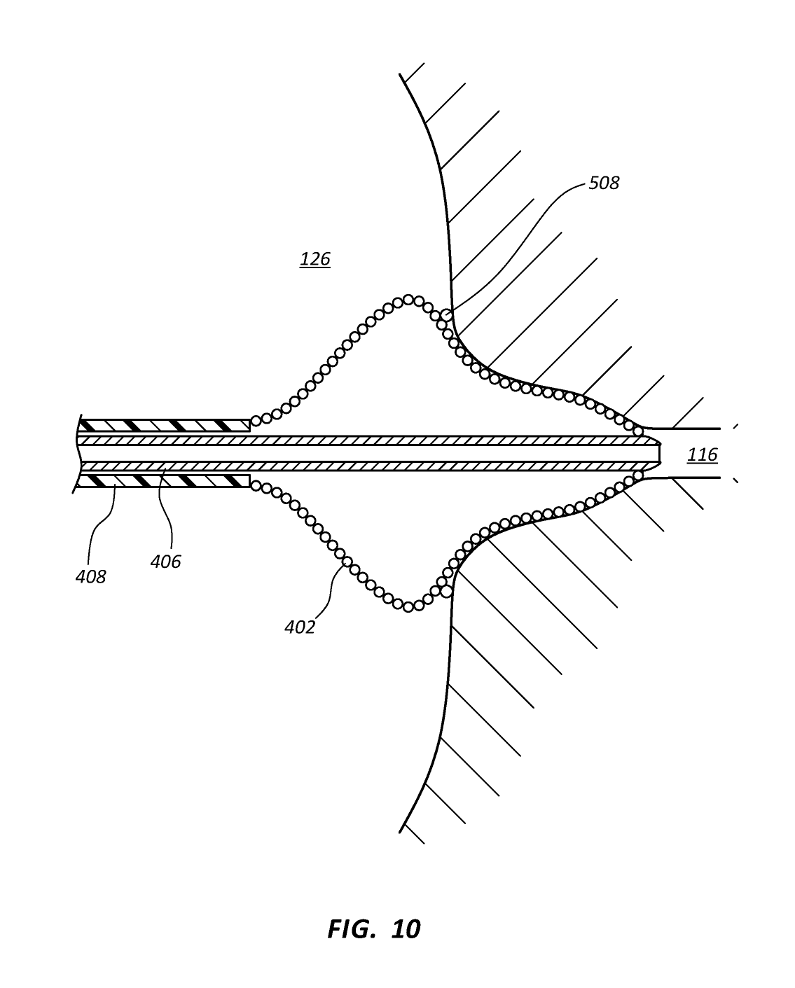

FIG. 10 illustrates the ablation device of FIG. 4 ablating a wall 1000 proximate the pulmonary vein 116. The distal end of the expandable mesh 402 is fitted within the pulmonary vein 116 centering the ablation device 400, in some embodiments the distal end of the inner shaft may extend beyond the flexible mesh 402 to form an elongated tip. The elongated tip may be used to center the location device in the vessel. The outer shaft 408 has been advanced relative to the inner shaft 406 expanding the expandable mesh 402. The expandable mesh 402 conforms to the shape of the ostium of the pulmonary vein 116. The flexible conductive coating is pressed against the wall and contacts the tissue. The flexible conductive coating 508 is in electrical communication with a first pole of a power source. A second pole of the power source is in electrical communication with the patient's body. Energy is delivered to the two poles and ablates the tissue proximate the flexible conductive coating 502 as it flows from the first pole to the second pole.

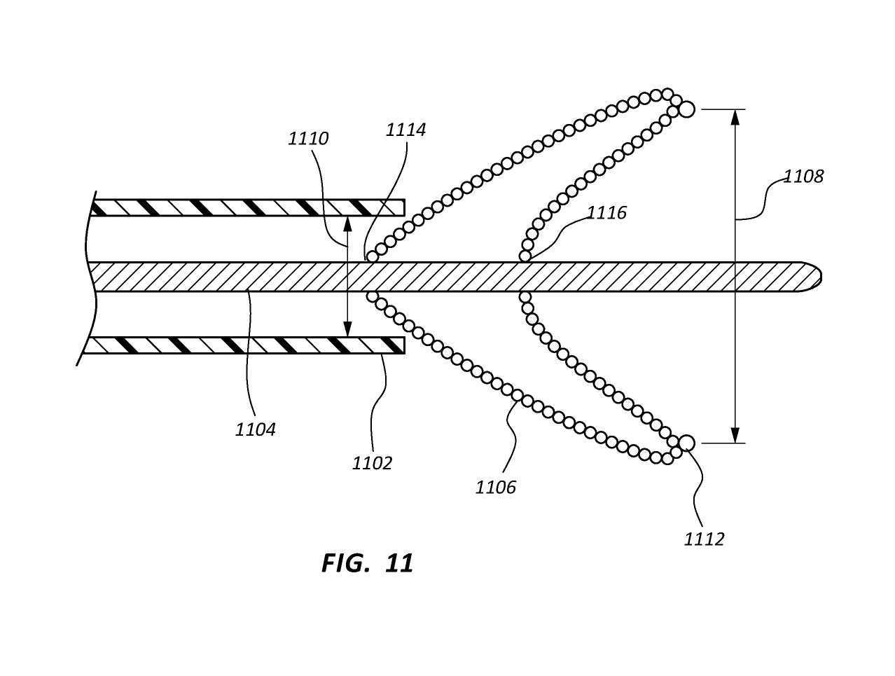

FIG. 11 shows another embodiment of an ablation device 1100. The ablation device comprises a shaft 1102, a sheath 1104, and an expandable mesh 1106. In this embodiment the expandable mesh 1106 is self-biased to the shape of a cone having a base diameter 1108 greater than an inside diameter 1110 of the sheath 1104. In one embodiment the expandable mesh 1106 is comprised of a material having shape memory. The expandable mesh is formed with the self-biased shape being a low energy state. The expandable mesh 1106 is connected to the shaft 1102 at an apex 1114 of the cone and at an apex 1116 of the interior of the cone. A flexible conductive coating 1112 is applied to the base of the cone and is in electrical communication with an energy source. The ablation device 1100 is illustrated in an expanded state, but may be compacted by moving the inner shaft proximally relative to the sheath so that the sheath covers the expandable mesh. The sheath provides a radial constraint that inhibits the cone from expanding to its self-biased conical shape.

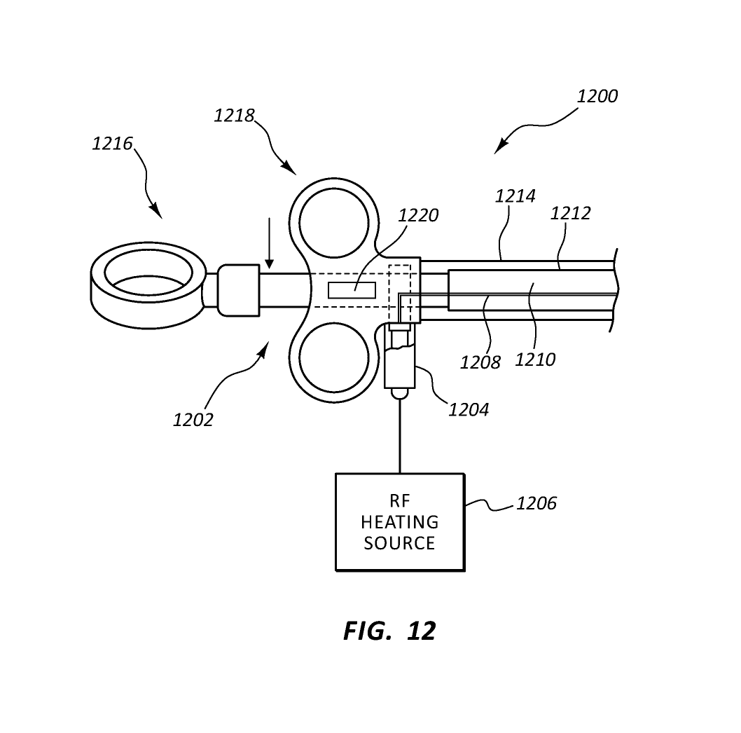

FIG. 12 illustrates the proximal end of an ablation device 1200. In each of the previously described embodiments, the conductive coating is operably connected to an energy source. As shown in FIG. 12, a handle 1202 may include a connector 1204 for operably connecting the conductive coating to an energy source 1206. As shown, the energy source 1206 may be a radio frequency source. However, other types of energy sources may also be used to provide energy to the conductive coating. By way of non-limiting example, additional possible energy sources may include microwave and electric current. The energy source 1206 may incorporate feedback such as temperature and impedance measurements to control the energy delivered to the ablation device 1200 during use.

The conductive coating is connected to the energy source 1206 by an electrical conductor, such as one or more wires 1208 that extend from the conductive coating to the connector 1204 that connects to the energy source 1206. The one or more wires 1208 may extend through a lumen 1210 of the inner shaft 1212 or may extend through a lumen of the outer shaft 1214 or external to the outer shaft 1214 and may optionally include a sleeve surrounding the outer shaft 1214 and one or more wires 1208.

As discussed above, the handle 1202 is operable to move the inner shaft 1212 relative to the outer shaft 1214 so that the expandable mesh 1202 moves between the expanded configuration and the collapsed configuration. By way of non-limiting example, the handle 1202 includes a first portion 1216 and a second portion 1218 that move relative to each other. As shown in FIG. 12, the first portion 1216 is operably connected to the inner shaft 1212. The second portion 1218 is operably connected to the outer shaft 1214. The first portion 1216 may be moved proximally and/or the second portion 1218 may be moved distally to move the inner shaft 1212 proximally and/or the outer shaft 1214 distally to move the expandable mesh 402 to the expanded configuration. The first portion 1216 may be moved distally and/or the second portion 1218 moved proximally to move the inner shaft 1212 distally and/or the outer shaft 1214 proximally to move the expandable mesh 402 to the collapsed configuration.

The handle 1202 may include a lock 1220 shown to releasably lock the first portion 1216 in position relative to the second portion 1218 and thus lock the expandable mesh 402 in position. The lock 1220 may releasably lock the first and second portions 1216, 1218 of the handle 1202 together at any proximal/distal positioning of the inner and outer shafts 1212, 1214 so that the expandable mesh 402 may be locked at any size that is suitable for the treatment site.

The above Figures and disclosure are intended to be illustrative and not exhaustive. This description will suggest many variations and alternatives to one of ordinary skill in the art. It is contemplated that the different described embodiments may be combined with one another. For example, the bipolar configuration of FIG. 9 is suitable for use in the example shown in FIG. 10 and similarly the bipolar configuration of FIG. 9 may have alternative expanded mesh configurations such as those shown in FIGS. 7, 10, and 11. All such variations and alternatives are intended to be encompassed within the scope of the attached claims. Those familiar with the art may recognize other equivalents to the specific embodiments described herein which equivalents are also intended to be encompassed by the attached claims.

What is claimed:

1. A medical device for ablating tissue, the medical device comprising:
a first longitudinal member having a first longitudinal member distal end;
a second longitudinal member having a second longitudinal member distal end, the second longitudinal member axially movable from a proximal position to a distal position;
a mesh having a mesh distal end attached to the second longitudinal member and a mesh proximal end attached to the first longitudinal member, the mesh comprised of a plurality of flexible non-conductive filaments woven together, the mesh being expandable from an unexpanded configuration to an expanded configuration by axially moving the first longitudinal member and the second longitudinal member relative to each other; and
a conductive coating on an outer surface of the mesh.

2. The medical device of claim 1, further comprising an energy source in electrical communication with the conductive coating.

3. The medical device of claim 1, wherein the conductive coating comprises a conductive ink printed on the outer surface of the cylindrical mesh.

4. The medical device of claim 1, further comprising a base material disposed in a circumferential portion about the mesh, wherein the conductive coating comprises a conductive ink printed on the base material.

5. The medical device of claim 4, wherein the base material comprises silicone.

6. The medical device of claim 1, further comprising a second conductive coating axially offset from the conductive coating and a circumferential non-conduction portion disposed between the conductive coating and the second conductive coating.

7. The medical device of claim 6, further comprising a bipolar energy source having a first pole in electrical communication with the conductive coating and a second pole in electrical communication with the second conductive coating.

8. The medical device of claim 1, wherein the mesh is cone shaped and the conductive coating is disposed on a base of the cone shape.

9. The medical device of claim 1, wherein the second longitudinal member extends distally beyond the mesh.

10. The medical device of claim 1, wherein the first longitudinal member has longitudinal lumen and the second longitudinal member is disposed within the longitudinal lumen, wherein the mesh distal end is connected to a distal end of the first longitudinal member distal end and the mesh proximal end is connected to the second longitudinal member distal end.

11. A medical device for ablating tissue, the medical device comprising:
a sheath having a longitudinal lumen with an inside diameter;
a shaft disposed within the longitudinal lumen of the sheath, the shaft being moveable relative to the lumen from a proximal position to a distal position;
a mesh attached to a distal end of the shaft, the mesh self-biased to a shape having a mesh outside diameter greater than the inside diameter of the sheath and comprising a plurality of flexible non-conductive filaments; and
a conductive coating disposed on an outer surface of the mesh.

12. The medical device of claim 11, wherein the mesh is biased to a cone shape having a base diameter greater than the inside diameter.

13. The medical device of claim 12 wherein the coating is disposed on a base of the cone shape.

14. The medical device of claim 11, wherein the shaft extends distally beyond the mesh.

15. The medical device of claim 11, further comprising a base layer disposed between the coating and the mesh.

16. The medical device of claim 15, wherein the base layer comprises silicone.

17. The medical device of claim 11, wherein the conductive coating comprises a conductive ink.

18. The medical device of claim 11, further comprising an energy source in electrical communication with the conductive coating.

19. The medical device of claim 11, further comprising a second conductive coating axially offset from the conductive coating.

20. The medical device of claim 19, further comprising an energy source having a first pole and a second pole, the first pole in electrical communication with the coating and the second pole in electrical communication with the second coating.
